(19) United States
(12) Patent Application Publication
(10) Pub. No.: US 2008/0262089 A1
(43) Pub. Date: Oct. 23, 2008

(54) Process for the Preparation of Tamsulosin

(75) Inventors: Jose Espinos Taya, Barcelona (ES); Ignasi Auquer Pedemonte, Girona (ES)

Correspondence Address: HOGAN & HARTSON LLP;IP GROUP, COLUMBIA SQUARE, 555 THIRTEENTH STREET, N.W., WASHINGTON, DC, 20004, US

(73) Assignee: MEDICHEM, S.A., Barcelona, ES (ES)

(21) Appl. No.: 11/913,652

(22) PCT Filed: May 4, 2006
(86) PCT No.: PCT/IB2006/002654

§ 371 Date: Jun. 24, 2008

Related U.S. Application Data
(60) Provisional application No. 60/677,339, filed on May 4, 2005.

(30) Foreign Application Priority Data
May 4, 2006 (IB) PCT/IB2006/002654

(51) Int. Cl. A61K 31/18; C07C 303/40; C07C 311/37

(52) U.S. Cl. 514/603; 564/86

(57) ABSTRACT

The invention includes an improved process for producing tamsulosin comprising reacting 5-(2-aminopropyl)-2-methoxybenzenesulfonamide with 2-(o-ethoxyphenoxy)ethyl bromide in an organic phosphite solvent to obtain tamsulosin. Optically pure (R)-5-(2-aminopropyl)-2-methoxybenzenesulfonamide can be employed to produce optically pure (R)-tamsulosin product. The organic phosphite solvent utilized in the reaction can include tri-alkyl phosphites such as triethyl phosphite, trimethyl phosphite, and tributyl phosphite. Additionally, processes for producing tamsulosin having a low concentration of by-product contaminants, such as 5-((R)-2-{Bis-[2-(2-ethoxyphenoxy)ethyl]amino}-propyl)-2-methoxybenzenesulfonamide, and the use of such by-products to monitor the chemical purity of tamsulosin, are provided.

### CROSS REFERENCE TO RELATED APPLICATIONS

This application claims priority to U.S. Provisional Application No. 60/677,339, filed May 4, 2005, which is expressly incorporated herein by reference in its entirety.

### BACKGROUND OF THE INVENTION

1. Field of the Invention

The invention relates, in general, to the preparation of tamsulosin. More particularly, the invention relates to the preparation of tamsulosin in a simplified process that provides a maximum yield of desired product with a minimum amount of undesired by-products. The invention further includes formulating tamsulosin, its salts and/or in vivo cleavable prodrugs thereof (collectively “the compounds of the invention”) into readily usable dosage units for the therapeutic treatment (including prophylactic treatment) of mammals, including humans.

2. Discussion of the Related Art

Tamsulosin hydrochloride is a commercially marketed pharmaceutically active substance known to be useful for the treatment of prostatic disorders, such as benign prostatic hyperplasia, as it is believed to operate as an antagonist of alpha1A adrenoceptors in the prostate. Tamsulosin hydrochloride, having an empirical formula of C₂₀H₂₈N₂O₅S.HCl and a molecular weight (“MW”) of 444.98, can be readily obtained from tamsulosin. Tamsulosin is the international common accepted name for 5-[(2R)-2-[[2-(2-ethoxyphenoxy)ethyl]amino]propyl]-2-methoxybenzene-sulfonamide, which is represented in formula (I).

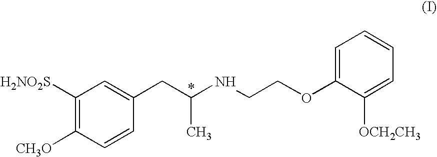

Notably, as depicted in formula (I), tamsulosin appears in two enantiomeric forms (R and S), with the R form being the more commercially desirable enantiomer due to its pharmacological activity.

There are various known mechanisms for producing tamsulosin. For example, U.S. Pat. No. 4,731,478 (the “'478 patent”) describes several processes by which tamsulosin can be produced. A first process of the '478 patent includes the conversion of a hydroxylated analogue into the desired sulfonamide via a chloro-analogue. The hydroxy analogue of tamsulosin is a compound having a structure as depicted in formula (II).

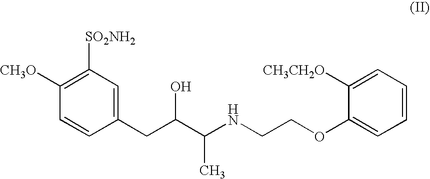

A second process comprises a reductive amination of a benzylmethylketone compound with an appropriately substituted phenoxyethylamine. For making tamsulosin according to this process, the benzylmethylketone can be represented by formula (III), and the phenoxyethylamine can be represented by formula (IV).

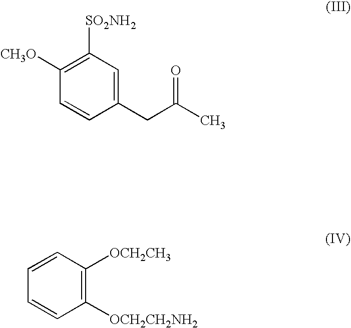

Unfortunately, these two processes described above produce racemic (R, S) tamsulosin, requiring the additional step of isolation of the R enantiomer form if the product is to be used for pharmaceutical purposes. While optical purification of racemic tamsulosin is possible, it is generally undesirable commercially.

The '478 patent, therefore, describes a process capable of producing optically pure enantiomer forms of tamsulosin. This process includes obtaining optically pure 5-(2-aminopropyl)-2-methoxy benzenesulfonamide, or 5-((2-amino-2-methyl)ethyl)-2-methoxybenzenesulfonamide, as depicted in formula (V), and reacting it with 2-(o-ethoxy phenoxy)ethyl bromide, as depicted in formula (VI), to form the corresponding tamsulosin enantiomer.

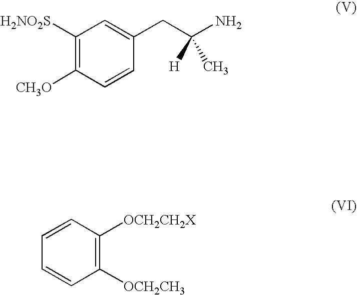

Thus (R)-5-(2-aminopropyl)-2-methoxybenzenesulfonamide) can be used to produce (R)-tamsulosin. In particular, this process for preparing optically pure tamsulosin is described in the '478 patent as mixing the starting compounds in ethanol where they react at reflux temperature for about 16 hours to produce a crude oily tamsulosin product. The crude oily tamsulosin product includes unused reactants and undesirable by-products of the reaction. This crude oily product thereafter requires a purification procedure by means of silica-gel column chromatography to isolate the tamsulosin before it can be converted into the desired corresponding hydrochloride.

European patent application EP 380 144 A (the “'144A application”) describes a similar process for producing optically pure tamsulosin that reacts compounds of formula (V) and formula (VI) at either room temperature, elevated temperature, or under reflux and either in the absence of solvent or in an organic solvent, such as benzene, toluene, xylene, dimethylformamide, dichloromethane, methanol or ethanol. The '144A application teaches that, optionally, secondary or tertiary amines (e.g., pyridine, picoline, N,N-dimethylaniline, N-methylmorpholine, trimethylamine, triethylamine or dimethylamine) or inorganic bases (e.g., potassium carbonate, sodium carbonate or sodium bicarbonate) can additionally be used to ensure a smooth reaction. As with the process described in the '478 patent, this process requires purification of crude tamsulosin product before thereafter further converting it into tamsulosin hydrochloride.

These known processes for producing tamsulosin, and, in particular, (R)-tamsulosin, are not optimal for industrial implementation because they lead to a presence of high amounts of undesired by-products which in turn makes it necessary to use economically disadvantageous purification processes to isolate the product to the extent required by quality specifications, such as to pharmaceutical grade product. Thus, there remains a need for improved processes for producing tamsulosin.

### SUMMARY OF THE INVENTION

The invention provides an improved method for producing tamsulosin, and, in particular, optically pure (R)-tamsulosin. This process according to the invention comprises reacting 5-(2-aminopropyl)-2-methoxybenzenesulfonamide, as depicted in formula (V) (also referred to herein as “reactant-V”), with 2-(o-ethoxyphenoxy)ethyl bromide, as depicted in formula (VI) (also referred to herein as “reactant-VI”), in an organic phosphite solvent to obtain tamsulosin. Preferably, optically pure (R)-5-(2-aminopropyl)-2-methoxybenzenesulfonamide) is employed as reactant-VI to produce optically pure (R)-tamsulosin product. Reactant-V can optionally be employed as the free base or as an additional salt with an organic or inorganic acid.

Additional embodiments of the invention include utilizing the tamsulosin product of the above reaction to further produce pharmaceutically acceptable or desirable additional salts, hydrates, solvates, or clathrates of tamsulosin. Preferably, these additional embodiments include forming an acid addition salt of tamsulosin obtained from the above reaction by treating a solution or suspension of that tamsulosin with an appropriate acid. Such processes isolate the tamsulosin product, such as by drying, boiling, and/or heating the product of the reaction in an organic solvent, and then reacting the isolated tamsulosin product to produce the tamsulosin salt, hydrate, solvate or clathrate.

Optionally, additional purification steps can be included without altering the invention, such as heating in an organic solvent, filtering, drying, and re-crystallization in a hot alkanol, such as ethanol.

Additionally, another embodiment of the invention further comprises monitoring the reaction products obtained from the reaction of reactant-V and reactant-VI for the presence of undesirable by-products. Preferably, such embodiments of the invention include monitoring the products for the presence of 5-((R)-2-{Bis-[2-(2-ethoxy-phenoxy)-ethyl]-amino}-propyl)-2-methoxy-benzenesulfonamide, which by-product is represented by structural formula (VII).

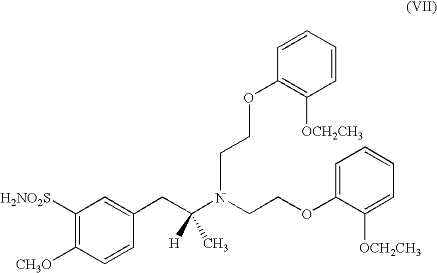

The presence of this by-product can be reduced or avoided by various mechanism. None, however, are as attractive as the process according to the invention. For example, one or more complicated separation processes, such as column chromatography, can be run on the impure tamsulosin product obtained from prior art methods in order to obtain high grade product. Such separation processes, however, are generally undesirable as they do lend well to large scale production. Similarly, it has been found that utilizing large excesses of optically pure (R)-reactant-V decreases the amount of by-product. This, however, requires the efficient separation and recovery of the reactant in order to avoid waste.

Processes according to the invention enable high quality and purity tamsulosin to be produced without requiring complicated separation procedures, such as column chromatography. In this regard, the processes of the invention enable the production of tamsulosin product having less than 0.05% area by HPLC of the by-product of formula (VII) without requiring procedures to separate that by-product from crude tamsulosin reaction product.

The invention further includes formulating tamsulosin, its salts and/or in vivo cleavable prodrugs thereof (collectively “the compounds of the invention”) into readily usable dosage units for the therapeutic treatment (including prophylactic treatment) of mammals, including humans.

The various embodiments of the invention having thus been generally described, several examples will hereafter be discussed to illustrate the inventive aspects more fully.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention. In the drawings:

FIG. 1 illustrates the X-ray powder diffractogramn of tamsulosin HCl obtained in Example 3.

### DETAILED DESCRIPTION OF THE PREFERRED EMBODIMENTS

Reference will now be made in detail to the preferred embodiments of the invention. This invention may, however, be embodied in many different forms and should not be construed as limited to the embodiments set forth herein. In addition and as will be appreciated by one of skill in the art, the invention may be embodied as a method, system or process.

The invention provides an improved method for producing tamsulosin, and, in particular, optically pure (R)-tamsulosin. This process according to the invention comprises reacting 5-(2-aminopropyl)-2-methoxybenzenesulfonamide, as depicted in formula (V) (also referred to herein as “reactant-V”), with 2-(o-ethoxyphenoxy)ethyl bromide, as depicted in formula (VI) (also referred to herein as “reactant-VI”), in an organic phosphite solvent to obtain tamsulosin. Preferably, optically pure (R)-5-(2-aminopropyl)-2-methoxybenzenesulfonamide) is employed as reactant-VI to produce optically pure (R)-tamsulosin product. Reactant-V can optionally be employed as the free base or as an additional salt with an organic or inorganic acid.

Notably, the starting materials, namely reactant-V (and its enantiomeric (R)-form) and reactant-VI are commercially available. Alternatively, of course, optically pure (R)-reactant-V can be obtained, for example, by treating racemic reactant-V with a sulfonic chiral acid such as (1R)-(−)-10-camphorsulphonic acid in an alkanol, preferably in a mixture of alkanol and water. The precipitated salt substantially enriched by the desired enantiomer would then be re-crystallized from a mixture of alkanol-water and purified to liberate the enriched enantiomer salt form. The liberation step can comprise treatment of the salt (in solid, suspended or dissolved state) with an organic or inorganic base. The base should be stronger than the basicity of desired enantiomer. Generally, the liberation of the desired enantiomer from the enriched salt proceeds by contacting the salt with an equivalent of a suitable base, e.g., metal hydroxides or ammonia, in a proper solvent, preferably in water. The free base of the desired enantiomer formed in this manner normally then can be isolated by ordinary methods. If water has been employed as solvent for neutralization, the desired enantiomer base would precipitate as a solid that can be isolated by filtration or centrifugation. The desired enantiomer base, can be treated again with a sulfonic chiral acid in order to increase its optically purity if needed.

In embodiments of the invention, the organic phosphite solvent utilized in the reaction includes tri-alkyl phosphites such as triethyl phosphite (MW 166.2; boiling point 153-157° C.), trimethyl phosphite (MW 124.1; boiling point 110-112° C.), and tributyl phosphite (MW 250.3; boiling point 125-128° C.). Preferably, the solvent is triethyl phosphite. The amount of organic phosphite solvent can range from approximately 65% to approximately 350% by weight with respect to the weight of reactant-V present, and preferably about be 320% by weight of the weight of reactant-V present.

In embodiments of the invention, reactant-V and reactant-VI are reacted in the organic phosphite solvent at an elevated temperature, preferably in the range of about 100° C. to about 160° C., and more preferably in the range of about 140° C. to about 150° C. Reaction time can vary with temperature, and can range from about 1 hour to about 8 hours, and more typically and preferably from about 2 hours to about 4 hours.

Additional embodiments of the invention include utilizing the tamsulosin product of the above reaction to further produce pharmaceutically acceptable or desirable additional salts, hydrates, solvates, or clathrates of tamsulosin. Preferably, these additional embodiments include forming an acid addition salt of tamsulosin obtained from the above reaction by treating a solution or suspension of that tamsulosin with an appropriate acid. Such processes isolate the tamsulosin product, such as by drying, boiling, and/or heating the product of the reaction in an organic solvent, and then reacting the isolated tamsulosin product to produce the tamsulosin salt, hydrate, solvate or clathrate.

Optionally, additional purification steps can be included without altering the invention, such as heating in an organic solvent, filtering, drying, and re-crystallization in a hot alkanol, such as ethanol.

It is preferred that in the compound of formula (VI), X is bromine and that the compound of formula (V) is used as its addition salt with an acid selected from hydrochloric acid, hydrofluoric acid, hydrobromic acid, hydroiodic acid, methanesulfonic acid, trifluoromethanesulfonic acid and/or trifluoroacetic acid, more preferably hydrochloric acid.

In embodiments of the invention, a neutralizing agent can be used to neutralize the hydrohalic acid which is formed in the coupling reaction. The neutralizing agent can be an organic or inorganic base, preferably selected from the group comprising alkali or alkaline earth metal carbonates, such as sodium carbonate or potassium carbonate, bicarbonates such as sodium bicarbonate, or tertiary amines such as triethylamine or diisopropylethylamine. Preferably, the neutralizing agent is sodium bicarbonate.

Preferably the neutralizing agent is used in excess. More preferably the process of the invention involves the use of from one to three molar equivalents of a neutralizing agent based on the starting material(s).

Additionally, another embodiment of the invention further comprises monitoring the reaction products obtained from the reaction of reactant-V and reactant-VI for the presence of undesirable by-products. Preferably, such embodiments of the invention include monitoring the products for the presence of 5-(R)-2-{Bis-[2-(2-ethoxy-phenoxy)-ethyl]-amino}-propyl)-2-methoxy-benzenesulfonamide, which by-product is represented by structural formula (VII).

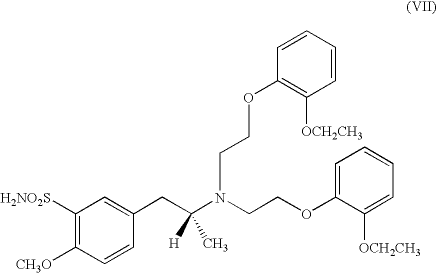

The presence of this by-product can be reduced or avoided by various mechanism. None, however, are as attractive as the process according to the invention. For example, one or more complicated separation processes, such as column chromatography, can be run on the impure tamsulosin product obtained from prior art methods in order to obtain high grade product. Such separation processes, however, are generally undesirable as they do lend well to large scale production. Similarly, it has been found that utilizing large excesses of optically pure (R)-reactant-V decreases the amount of by-product. This, however, requires the efficient separation and recovery of the reactant in order to avoid waste.

Processes according to the invention enable high quality and purity tamsulosin to be produced without requiring complicated separation procedures, such as column chromatography. In this regard, the processes of the invention enable the production of tamsulosin product having less than 0.05% area by HPLC method 1 of the by-product of formula (VII) without requiring procedures to separate that by-product from crude tamsulosin reaction product.

The invention further includes formulating tamsulosin, its salts and/or in vivo cleavable prodrugs thereof (collectively “the compounds of the invention”) into readily usable dosage units for the therapeutic treatment (including prophylactic treatment) of mammals, including humans. Such formulations are normally formulated in accordance with standard pharmaceutical practice as a pharmaceutical composition. According to this aspect of the invention there is provided a pharmaceutical composition that comprises the compounds of the invention, as defined hereinbefore in association with a pharmaceutically acceptable diluent or carrier.

The compositions of the invention may be in a form suitable for oral use (e.g., as tablets, lozenges, hard or soft capsules, aqueous or oily suspensions, emulsions, dispersible powders or granules, syrups or elixirs), for topical use (e.g., as creams, ointments, gels, or aqueous or oily solutions or suspensions), for administration by inhalation (e.g., as a finely divided powder or a liquid aerosol), for administration by insufflation (e.g., as a finely divided powder) or for parenteral administration (e.g., as a sterile aqueous or oily solution for intravenous, subcutaneous, or intramuscular dosing or as a suppository for rectal dosing). For example, compositions intended for oral use may contain, for example, one or more coloring, sweetening, flavoring and/or preservative agents.

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as the sodium salt of benzoic acid, ethyl or propyl p-hydroxybenzoate), anti-oxidants (such as ascorbic acid), coloring agents, flavoring agents, and/or sweetening agents (such as sucrose, saccharine or aspartame).

Oily suspensions may be formulated by suspending the active ingredient in a vegetable oil (e.g., arachis oil, olive oil, sesame oil or coconut oil) or in a mineral oil (e.g., liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (e.g., sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedures well known in the art.

Compositions for administration by insufflation may be in the form of a finely divided powder containing particles of average diameter of, for example, 30 μm or much less, the powder itself comprising either active ingredient alone or diluted with one or more physiologically acceptable carriers such as lactose. The powder for insufflation is then conveniently retained in a capsule containing, for example, 0.2 to 2 mg of active ingredient for use with a turbo-inhaler device, such as is used for insufflation of the known agent sodium cromoglycate.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used, and the aerosol device is conveniently arranged to dispense a metered quantity of active ingredient.

The amount of a compound of this invention that is combined with one or more excipients to produce a single dosage form will necessarily vary depending upon the host treated and the particular route of administration. For example, a formulation intended for oral administration to humans will may contain, for example, from 0.2 to 2 mg of active ingredient compounded with an appropriate and convenient amount of excipients which may vary from about 5 to about 98 percent by weight of the total composition. Dosage unit forms will generally contain about 0.2 to 2 mg of an active ingredient.

The size of the dose for therapeutic or prophylactic purposes of the compounds of the invention will naturally vary according to the nature and severity of the conditions, the age and sex of the animal or patient, and the route of administration, according to well known principles of medicine. For example, the method may comprise at least one of an hourly administration, a daily administration, a weekly administration, or a monthly administration of one or more compositions described herein.

In addition to the compounds of the invention, the invention also includes solvates, pharmaceutically acceptable prodrugs, pharmaceutically active metabolites, and pharmaceutically acceptable salts of such compounds.

The term “solvate” refers to an aggregate of a molecule with one or more solvent molecules.

A “pharmaceutically acceptable prodrug” is a compound that may be converted under physiological conditions or by solvolysis to the specified compound or to a pharmaceutically acceptable salt of such compound.

A “pharmaceutically active metabolite” is a pharmacologically active product produced through metabolism in the body of a specified compound or salt thereof. Metabolites of a compound may be identified using routine techniques known in the art, and their activities determined using tests such as those described herein.

Prodrugs and active metabolites of a compound may be identified using routine techniques known in the art. Various forms of prodrugs are known in the art.

According to the invention, suitable methods of administering the therapeutic composition of the invention to a patient include any route of in vivo administration that is suitable for delivering the composition into a patient. The preferred routes of administration will be apparent to those of skill in the art, depending on the type of condition to be prevented or treated, and/or the target cell population. Preferred methods of in vivo administration include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, intraarticular administration, intraventricular administration, inhalation (e.g., aerosol), intracranial, intraspinal, intraocular, intranasal, oral, bronchial, rectal, topical, vaginal, urethral, pulmonary administration, impregnation of a catheter, and direct injection into a tissue.

It will be apparent to those skilled in the art that various modifications and variations can be made in the invention and specific examples provided herein without departing from the spirit or scope of the invention. Thus, it is intended that the invention covers the modifications and variations of this invention that come within the scope of any claims and their equivalents.

The following examples are for illustrative purposes only and are not intended, nor should they be interpreted to, limit the scope of the invention.

### Example 1

### Preparation of (R)-5-(2-aminopropyl)-2-methoxy benzenesulfonamide

The preparation of (R)-5-(2-aminopropyl)-2-methoxybenzenesulfonamide (reactant-V) is provided according to this example. A mixture of racemic (±)-5-(2-aminopropyl)-2-methoxybenzenesulfonamide (7.9 g, 32.33 mmol) and (1R)-(−)-10-camphorsulfonic acid (7.5 g, 32.28 mmol) is prepared in isopropanol (20 mL) and then stirred for 30 minutes. A precipitate forms and is separated by filtration and then washed with a mixture of isopropanol and water. The obtained precipitated solid is crystallizated from a mixture of isopropanol and water and dried to obtain 4.3 g of salt.

The obtained salt (4.3 g) is then dissolved in water (25 mL), and the pH is adjusted with ammonia to 10 and then stirred for 1 hour. A precipitate forms and is then separated by filtration, washed with water and dried yielding (R)-5-(2-aminopropyl)-2-methoxybenzene sulfonamide (2 g, 8.18 mmol, 25.31% molar yield).

### Example 2

### Preparation of Tamsulosin Hydrochloride

Tamsulosin hydrochloride is prepared according to this example. In a round-bottomed flask, 4.8 g (19.65 mmol) of (R)-5-(2-aminopropyl)-2-methoxybenzenesulfonamide or (reactant-V), 16 mL (15.28 g, 91.96 mmol) of triethyl phosphite and 2.8 g (33.33 mmol) of sodium bicarbonate are charged. The suspension is stirred until complete solution at which point, 5.8 g (23.64 mmol) of 2-(o-ethoxyphenoxy)ethyl bromide (reactant-VI) are charged.

The mixture is stirred at reflux temperature for two hours. After this time, 16 mL of water is charged into the flask and the mixture is stirred at reflux temperature for an additional four hours. The mixture is thereafter cooled down to 0° C. and filtered.

The filtrate is alkalinized with concentrated ammonia until pH 9 and the resulting suspension is heated up to 40° C. and stirred for one hour. The suspension obtained is then again cooled down to 0° C.

Then, 30 mL of water and 75 mL of ethyl acetate (“AcOEt”) are also charged into the flask and the resulting mixture is heated up (to about 50° C.) to complete solution. The mixture is allowed to settle and the aqueous layer is separated, while keeping the temperature at about 50° C. The organic phase is further extracted twice with 100 and 75 mL of water at pH 6. The aqueous phases are combined and the pH is adjusted to pH 9 using diisopropylethylamine (“DIEA”) and tamsulosin base is thereafter extracted from the aqueous phase with 2×50 mL of AcOEt.

The solution is dried by evaporation, and the residue is treated with 69 mL of ethanol at 60° C., to obtain a clear solution. After cooling at 35-40° C., aqueous concentrate hydrochloric acid is added so as to adjust the pH to about 1-2, and the resulting mixture is cooled down to 0° C. Stirring is then maintained for 2 hours. A white solid precipitates that is then filtered, washed with ethanol, and dried at 60° C. in vacuum until constant weight, to yield 2.89 g (6.49 mmol, 33% molar yield) of tamsulosin hydrochloride. No by-product (VII) is detected by HPLC at this stage.

The tamsulosin hydrochloride thus obtained is further purified by treating with 29 mL of ethanol at 78° C., stirring for 30 minutes, cooling to 0° C., filtering the white solid that precipitates, washing with ethanol and drying at 60° C. in vacuum until constant weight to yield 2.66 g (5.98 mmol, 92% partial molar yield, 30.36% global yield) of tamsulosin hydrochloride. (Assay: 101.31%; IR: matches the standard; melting point: 228.8-229.6° C.; chemical purity: 99.58 area % by HPLC method 1; content of by-product (formula (VII)) by HPLC method 1: not detected).

### Example 3

### Preparation of Tamsulosin Hydrochloride

Tamsulosin Hydrochloride is also prepared according to this example by first repeating the initial reaction, cooling, and filtering steps of Example 2 to produce the filtrate at 0° C.

Instead of using ammonia, the filtrate is then alkalinized with DIEA until a pH of 8.5 is obtained, and the desired product is extracted with 2×50 mL of AcOEt. The combined organic layers are then extracted twice with 50 mL of water at pH 6 (adjusted with HCl). The aqueous phases are combined and the pH is again adjusted to 8.5 with DIEA, and the product is then extracted with 2×25 mL of AcOEt.

The combined organic phases obtained from the extractions are dried over Na₂SO₄ and the solvent is evaporated to obtain 3.28 g of crude tamsulosin. The content of byproduct as determined by HPLC method 1 was 0.261% (area percentage).

The residue is dissolved in ethanol (32 mL) and 1.65 mL (7.76 mmol) of ethanol HCl 4.7 N are charged. Then, the mixture is cooled down to 0° C. and the solid is collected by filtration, washed with ethanol and dried in vacuum at 60° C. until constant weight to yield 2.17 g (4.87 mmols, 26% molar yield) of tamsulosin hydrochloride. By subsequent analysis, the content of by-product (formula (VII)) by HPLC method 1 at this stage is 0.08 area %.

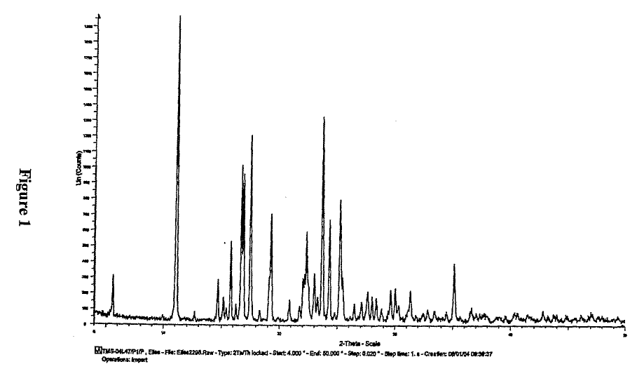

The tamsulosin hydrochloride thus obtained is further purified by repeating twice the following procedure: treating with 29 mL of ethanol at 78° C., stirring for 30 minutes, cooling to 0° C., filtering, washing with ethanol and then drying at 60° C. in vacuum until constant weight. After drying, 1.98 g of tamsulosin hydrochloride are obtained (4.45 mmols, 91% partial molar yield). (Assay: 100.21%; IR: matches the standard; melting point: 227.4-229.3° C.; chemical purity: 99.31 area % by HPLC method 1; XRD (2θ), see FIG. 1; content of by-product (formula (VII)) by HPLC method 1: 0.02 area %)

### Example 4

### Preparation of By-Product (5-((R)-2-{Bis-[2-(2-ethoxy phenoxy)ethyl]amino}-propyl)-2-methoxy benzene sulfonamide)

This example demonstrates how by-product 5-((R)-2-{Bis-[2-(2-ethoxyphenoxy)ethyl]amino}-propyl)-2-methoxybenzenesulfonamide is prepared by reacting the (R)-enantiomer of reactant-V according to prior processes. A round-bottomed flask is charged with 4.0 g (16.37 mmol) of (R)-reactant-V, 3.47 g (32.74 mmol) of Na₂CO₄, 8.01 g (32.65 mmol) of the bromide form of reactant-VI, and 24 mL of N,N-dimethylformamide. Notably, alkyl phosphite solvent was not used in this example. The mixture is heated up to 80° C., and is stirred overnight at 80° C., and then the mixture is cooled down to about 20-25° C.

At this point, 40 mL of water and 40 mL of AcOEt are charged into the flask and the resulting mixture is then stirred for 30 minutes and left to decant. The organic phase is separated and then charged again into the flask. is reintroduced in the flask and washed with slightly acidic water (pH 5). The mixture is let to decant and the organic phase is dried with Na₂SO₄ and the solvent evaporated to obtain 8.96 g of crude 5-((R)-2-{Bis-[2-(2-ethoxyphenoxy)ethyl]amino}-propyl)-2-methoxybenzene sulfonamide. The crude product was purified by silica gel column chromatography (AcOEt as eluent) to obtain 6.29 g of 5-((R)-2-{Bis-2-(2-ethoxyphenoxy)ethyl]amino}-propyl)-2-methoxybenzenesulfonamide, which still was unpurified and contained unused reactant-VI. A second silica gel column chromatography (CHCl₃ as eluent) was performed to obtain 6.00 g of purified 5-((R)-2-{Bis-[2-(2-ethoxyphenoxy)ethyl]amino}-propyl)-2-methoxybenzenesulfonamide (yield 64%).

Prior to purification by gel column chromotography, the crude product has approximately 9% (area) tamsulosin free base and approximately 73.3% (area) of the byproduct. Following purification by gel column chromotography, tamsulosin free base is not detected according to HPLC method 1 analysis.

The ¹H-NMR (DMSO-d6, 300 MHz), δ (ppm) of the byproduct is characterized as follows: 0.94 (d, 3H, NCHCH₃); 1.24 (t, 6H, OCH₂CH₃); 2.49 (m (overlapped with DMSO-d5), 1H, Ar—CHA); 2.84 (dd, 1H, Ar—CHB); 2.89-3.05 (complex signal, 5H, N(CH₂—)₂ and NH—CHCH₃); 3.83 (s, 3H, OCH₃); 3.78-4.01 (complex signal, 8H, 2OCH₂CH₃ and 2 NHCH₂CH₂O); 6.79-6.94 (complex signal, 8H, Ar—H of Ar—OEt); 6.97 (broad s, 2H SO₂NH₂); 7.00, (d, 1H, 3-H (Ar—SO₂NH₂)); 7.41 (dd, 1H, 4-H (Ar—SO₂NH₂); 7.57 (d, 1H, 6-H (Ar—SO₂NH₂));

The ¹³C NMR (DMSO-d6, 300 MHz), δ (ppm) of the byproduct is characterized as follows: 14.9 (2CH₃, 2OCH₂CH₃); 15.3 (CH₃, CHCH₃); 38.3 (CH₂, ArCH₂); 50.1 (2CH2, 2 OCH₂CH₂N); 56.1 (CH₃, OCH₃); 59.2 (CH, CHCH₃); 63.9 (2CH₂, 2OCH₂CH₃); 68.6 (2CH₂, 2OCH₂CH₂N); 112.4 (CH, C₃ (Ar—SO₂NH₂)); 113.6, 113.7 and 120.9 (2×4CH (Ar—OEt)); 128.1 (CH, C₆ (Ar—SO₂NH₂); 130.9 and 132.3 (2×C, C₁ and C₅ (Ar—SO₂NH₂)); 134.3 (CH, C4 (Ar—SO₂NH₂); 148.4 and 148.5 2×2C (Ar—OEt); 154.2 (C, C2 (Ar—SO₂NH₂).

### Reference Example

This example describes the results of a reaction similar to that of the examples above, which was performed using the same starting materials but without using an alkyl phosphite as a reactant and solvent. In a round-bottomed flask 3.0 g (12.28 mmols) of (R)-reactant-V, 12 mL of N,N-dimethylformamide and 2.55 mL (1.79 g, 14.92 mmols) of N,N-diisopropylethylamine are charged. The suspension is stirred until complete solution and then, 2.86 g (11.66 mmols) of 2-(o-ethoxyphenoxy)ethyl bromide, reactant-VI, are charged. The mixture is heated to 100° C. and kept for 1 hour. The reaction is checked for completion by thin layer chromatography. After cooling to room temperature, 15 mL of water and 36 mL of AcOEt are added. By decanting, filtering, and drying in fashion similar to that described in the previous examples, tamsulosin free base is obtained, which is then converted to tamsulosin hydrochloride. Content of the by-product represented by formula (VII) is found by HPLC method 1 be 0.32% (area). After crystallizing the product with ethanol, the product obtained thereafter has a content of the byproduct by HPLC method 1 of 0.15%.

### Example 5

### Preparation of Enantiomerically Enriched (R)-5-(2-aminopropyl)-2-methoxybenzenesulfonamide

The enantiomeric enrichment of (R)-5-(2-aminopropyl)-2-methoxybenzenesulfonamide (reactant-V) is provided according to this example. In a round bottomed flask 20.88 g of (R)-5-(2-aminopropyl)-2-methoxybenzene-sulfonamide (85.46 mmol, e.e. 96.4% by HPLC method 2), 19.85 g of (1R)-(−)-10-camphorsulfonic acid (85.46 mmol), 310.86 g of isopropanol (396 mL) and 26.43 mL of water were charged. The resulting mixture was heated to reflux and was stirred for 30 minutes at this temperature; in this way, a yellowish homogeneous solution was obtained. This solution was cooled down to 2±2° C. and aged for 60 minutes. Then the off-white solid thus obtained was filtered under vacuum to yield 46.15 g of a wet solid (loss on drying: 24.4%, which corresponds to 34.88 g of dry material; Yield: 85.65%).

To the above obtained solid, 617.01 g of isopropanol and 26.19 mL of water were added, and the mixture was heated to reflux and was stirred for 10 minutes at this temperature. The thick suspension thus obtained was cooled down to 2±2° C., aged for 60 minutes and the resulting off-white solid was filtered under vacuum to yield 38.44 g of a wet solid (loss on drying: 10.43%, which corresponds to 34.43 g of dry material; Yield: 98.71%).

To the solid thus obtained, 207 mL of water were added and the mixture was stirred for 30 minutes at 20-25° C.; in this way, a yellowish homogeneous solution was obtained. Then 25.06 mL of aqueous ammonia were added to the solution to adjust the pH at 10.18, with continuous stirring and keeping the temperature at about 25° C., and a white solid was obtained. The mixture was concentrated by distillation under vacuum until 69.28 mL of water were distilled and a white solid precipitated out. Then 17.64 mL of aqueous ammonia at 25° C. were added to adjust the pH at 10.07. The obtained mixture was cooled down to 2±2° C. and aged for 60 minutes. The off-white solid thus obtained was filtered under vacuum and dried under vacuum at 60° C. to yield 14.65 g of (R)-5-(2-aminopropyl)-2-methoxybenzene-sulfonamide. (Yield: 82.06%, overall yield 69.4%, e.e. 100.0% by HPLC method 2)

### Example 6

### Preparation of Enantiomerically Enriched (R)-5-(2-aminopropyl)-2-methoxybenzenesulfonamide

The enantiomeric enrichment of (R)-5-(2-aminopropyl)-2-methoxybenzenesulfonamide (reactant-V) is provided according to this example. In a round bottomed flask 41.76 g of (R)-5-(2-aminopropyl)-2-methoxybenzene-sulfonamide (107.9 mmol, e.e. 96.4% by HPLC method 2), 170.7 g of (1R)-(−)-10-camphorsulfonic acid (107.9 mmol), 621.7 g of isopropanol (792 mL) and 53 mL of water were charged. The resulting mixture was heated to reflux and was stirred for 30 minutes at this temperature; in this way, a yellowish homogeneous solution was obtained. This solution was cooled down to 2±2° C. and aged for 60 minutes. Then the off-white solid thus obtained was filtered under vacuum to yield 97.44 g of a wet solid (loss on drying: 25.0%, which corresponds to 73.05 g of dry material; Yield: 89.67%).

To the above obtained solid, 1212 g of isopropanol (1544 mL) and 52 mL of water were added, and the mixture was heated to reflux and was stirred for 10 minutes at this temperature. The thick suspension thus obtained was cooled down to 2±2° C., aged for 60 minutes and the resulting off-white solid was filtered under vacuum to yield 78.97 g of a wet solid (loss on drying: 25.6%, which corresponds to 58.72 g of dry material; Yield: 81.36%).

To the solid thus obtained, 352 mL of water were added and the mixture was stirred for 30 minutes at 20-25° C.; in this way, a yellowish homogeneous solution was obtained. Then 42 mL of aqueous ammonia were added to the solution to adjust the pH at 9.89, with continuous stirring and keeping the temperature at about 25° C., and a white solid was obtained. The mixture was concentrated by distillation under vacuum until 166 mL of water were collected and a white solid precipitated out. Then 14 mL of aqueous ammonia at 25° C. were added to adjust the pH at 9.97. The obtained mixture was cooled down to 2±2° C. and aged for 60 minutes. The off-white solid thus obtained was filtered under vacuum to yield 28.72 g of a wet solid (loss on drying: 12.55%, which corresponds to 25.15 g of dry material; Yield: 83.57%, overall yield 61.0%, e.e. 99.44% by HPLC method 2).

To the solid thus obtained, 314.7 g of isopropanol (401 mL) were added and the mixture was heated to reflux and was stirred for 30 minutes at this temperature. The solution was concentrated by distillation under atmospheric pressure until 165 g of isopropanol (211 ml) were collected and a white solid precipitated out. The mixture was cooled down to 2±2° C. and aged for 60 minutes. The off-white solid thus obtained was filtered under vacuum and dried under vacuum at 60° C. to yield 20.98 g of (R)-5-(2-aminopropyl)-2-methoxybenzene-sulfonamide. (Yield: 83.5%, overall yield 50.86%).

Analysis: Potentiometric assay using HClO₄: 99.75%. Melting point: 169.3-170.2 enantiomeric excess, e.e 99.5% by HPLC method 2.

General Experimental Conditions:

In the various examples described above, two analytical chromatographic HPLC methods were used:

HPLC method 1: tests were carried out by reversed-phase ion-pair chromatography in a Kromasil C8 column of 5 μm and 250×4.6 mm using a gradient system. Mobile phases included a mobile phase B, consisting of acetonitrile and mobile phase A, prepared by mixing 300 mL of acetonitrile with 700 mL of buffer at a pH=3.5, which is prepared from 0.78 g KH₂PO₄ and 1.33 g of 1-pentanesulfonic acid sodium salt dissolved in 700 mL of water, adjusting the pH with H₃PO₄. This mobile phase was mixed and filtered through a 0.22 μm filter under vacuum.

The chromatograph was equipped with a 225 nm detector and the flow rate was 1.0 mL per minute at room temperature. The gradient was defined by the following points: 100% mobile phase A from initial conditions to 10 minutes, 80% mobile phase A in 10 minutes, maintain 80% mobile phase A for 30 minutes, 100% mobile phase A, initial conditions, in 10 minutes.

HPLC method 2 (Chiral): The chromatographic separation was carried out in a Chiralpack AD-H, 5 μm, 4.6×250 mm column; at 30° C.

The mobile phase A was prepared by mixing 200 ml of 0.1% diethylamine in 2-propanol with 800 ml of n-hexane. This mobile phase was mixed and filtered through 0.22 μm nylon filter under vacuum. The mobile phase B was 2-propanol.

The chromatograph was equipped with a 280 nm detector and the flow rate was 1 ml per minute of mobile phase obtained by mixing 450 ml of mobile phase A and 550 ml of mobile phase B. Test samples were prepared by dissolving the appropriate amount of sample to obtain 0.5 mg per ml of mixture of mobile phase and 30 μl were injected

Although the invention has been described and illustrated with a certain degree of particularity, it is understood that the disclosure has been made only by way of example, and that numerous changes in the conditions and order of steps can be resorted to by those skilled in the art without departing from the spirit and scope of the invention.

What is claimed is:

1. A process for producing tamsulosin, comprising:
reacting in an organic phosphite solvent a first compound of the formula 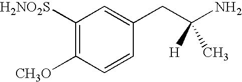
with a second compound of the formula 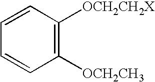
wherein X is a halogen.

2. The process according to claim 1, wherein said organic phosphite solvent is at least one of triethyl phosphite, trimethyl phosphate, tributyl phosphate and combinations thereof.

3. The process according to claim 2, wherein said organic phosphite solvent comprises triethyl phosphite.

4. The process according to claim 1, wherein said first compound is optically pure.

5. The process according to claim 4, wherein said first compound comprises 5-(2-aminopropyl)-2-methoxy benzenesulfonamide.

6. The process according to claim 1, wherein said organic phosphite solvent is present in an amount ranging from approximately 65% to approximately 350% by weight in comparison to the weight of the first compound.

7. The process according to claim 1, wherein said reaction takes place in the organic solvent at a temperature in the range of about 100° C. to about 160° C.

8. The process according to claim 7, wherein said reaction takes place in the organic solvent at a temperature in the range of about 140° C. to about 150° C.

9. The process according to claim 1 further comprising recovering tamsulosin from said solvent after an appropriate reaction time and converting said recovered tamsulosin into an addition salt, a hydrate, a solvate or a clathrate of tamsulosin.

10. The process according to claim 1 further comprising recovering tamsulosin from said solvent after an appropriate reaction time and converting said recovered tamsulosin into tamsulosin hydrochloride.

11. The process according to claim 1, wherein said halogen is bromine and said first compound is provided in the form of an additional salt with an acid selected from the group consisting of hydrochloric acid, hydrofluoric acid, hydrobromic acid, hydroiodic acid, methanesulfonic acid, trifluoromethanesulfonic acid and trifluoroacetic acid.

12. The process according to claim 1 further comprising neutralizing a halohalic acid formed by the reaction of said first compound and said second compound with a base.

13. The process according to claim 12, wherein said base is at least one of alkali or alkaline earth metal carbonates, alkali or alkaline earth metal bicarbonates, tertiary amines and combinations thereof.

14. The process according to claim 12, wherein said base is used in excess.

15. The process according to claim 1, wherein said produced tamsulosin having less than 0.05% area by HPLC of the by-product of the formula 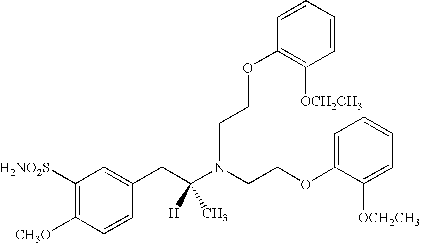
without requiring procedures to separate said by-product from a crude tamsulosin reaction product.

16. The process according to claim 1 further comprising
recovering tamsulosin from a crude tamsulosin product by at least one of extracting, filtering, drying and re-crystallizing.

17-23. (canceled)

24. The process according to claim 1 further comprising preparing a dosage unit comprising combining at least one adjuvant and at least one of tamsulosin and a salt of tamsulosin in a pharmaceutically deliverable form.

25. A compound of the formula 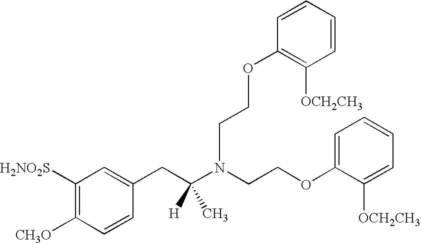
characterized by a ¹H-NMR (DMSO-d6, 300 MHz), δ (ppm) having its main peaks at approximately 0.94 (d, 3H, NCHCH₃); 1.24 (t, 6H, OCH₂CH₃); 2.49 (m (overlapped with DMSO-d5), 1H, Ar—CHA); 2.84 (dd, 1H, Ar—CHB); 2.89-3.05 (complex signal, 5H, N(CH₂—)₂ and NH—CHCH₃); 3.83 (s, 3H, OCH₃); 3.78-4.01 (complex signal, 8H, 2OCH₂CH₃ and 2 NHCH₂CH₂O); 6.79-6.94 (complex signal, 8H, Ar—H of Ar—OEt); 6.97 (broad s, 2H SO₂NH₂); 7.00, (d, 1H, 3-H (Ar—SO₂NH₂)); 7.41 (dd, 1H, 4-H (Ar—SO₂NH₂); and 7.57 (d, 1H, 6-H (Ar—SO₂NH₂)), and
a ¹³C NMR (DMSO-d6, 300 MHz), δ (ppm) having its main peaks at approximately 14.9 (2CH₃, 2OCH₂CH₃); 15.3 (CH₃, CHCH₃); 38.3 (CH₂, ArCH₂); 50.1 (2CH2, 2OCH₂CH₂N); 56.1 (CH₃ OCH₃); 59.2 (CH, CHCH₃); 63.9 (2CH₂, 2OCH₂CH₃); 68.6 (2CH₂, 2 OCH₂CH₂N); 112.4 (CH, C₃ (Ar—SO₂NH₂)); 113.6, 113.7 and 120.9 (2×4CH (Ar—OEt)); 128.1 (CH, C₆ (Ar—SO₂NH₂); 130.9 and 132.3 (2×C, C₁ and C₅ (Ar—SO₂NH₂)); 134.3 (CH, C4 (Ar—SO₂NH₂); 148.4 and 148.5 2×2C (Ar—OEt); and 154.2 (C, C2 (Ar—SO₂NH₂).

26-28. (canceled)

29. The process according to claim 1 further comprising monitoring the chemical purity of tamsulosin or pharmaceutical compositions comprising tamsulosin, said monitoring comprising:
using a compound of the formula 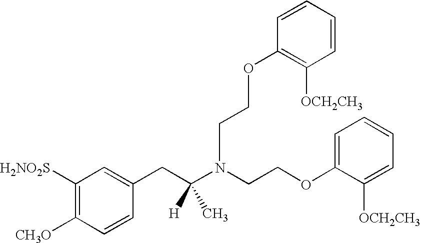
as a marker to monitor the chemical purity of tamsulosin.

30. The processes of any of claim 1 further comprising monitoring the reaction of said first compound and said second compound for the presence of 5-((R)-2-{Bis-[2-(2-ethoxyphenoxy)ethyl]amino}-propyl)-2-methoxybenzenesulfonamide.

31. A process for enantiomerically enriching (R)-5-(2-aminopropyl)-2-methoxybenzenesulfonamide or resolving a racemate thereof comprising:
producing a first precipitate in a solution of (±) 5-(2-aminopropyl)-2-methoxybenzenesulfonamide and a chiral sulfonic acid in at least one of an alkanol or a mixture of an alkanol and water;
isolating said first precipitate;
dissolving said first precipitate in water and adjusting the pH to approximately 10; and
producing a second precipitate, wherein said second precipitate is (R)-5-(2-aminopropyl)-2-methoxybenzenesulfonamide.

32. The process of claim 31, wherein said chiral sulfonic acid is (1R)-(−)-10-camphorsulfonic acid.

33. The process of claim 31, wherein said alkanol is isopropanol.

34. The process of claim 31 further comprising crystallizing said first precipitate.

35-39. (canceled)
